(19) United States
(12) Patent Application Publication
(10) Pub. No.: US 2020/0078075 A1
(43) Pub. Date: Mar. 12, 2020

(54) TREATMENT SYSTEM AND CONTROL APPARATUS

(71) Applicant: Olympus Corporation, Tokyo (JP)

(72) Inventor: Toshifumi Katsuragi, Tokyo (JP)

(73) Assignee: Olympus Corporation, Tokyo (JP)

(21) Appl. No.: 16/680,722

(22) Filed: Nov. 12, 2019

Related U.S. Application Data
(63) Continuation of application No. PCT/JP2017/018546, filed on May 17, 2017.

(51) Int. Cl. A61B 18/10; B06B 1/02; A61B 18/12

(52) U.S. Cl. CPC A61B 18/10; B06B 1/0284; A61B 2017/00973; B06B 2201/76; A61B 18/1206

(57) ABSTRACT

A treatment system includes a control apparatus having respective first and second output sources that supplies respective high-frequency power defined by a first electrical energy and a second electrical energy. The control apparatus includes a processor configured to acquire a first value based on the first electrical energy and a second value based on the second electrical energy. Based on the first value, the processor determines influence given to the second value by the first electrical energy. Based on the influence given to the second value, the processor adjusts an output instruction value to the second output source by the first electrical energy and the second value based on the second electrical energy. The control apparatus is configured to properly calculate a physical quantity relating to the first and second electrical energies even when an electrical noise is generated in a state in which multiple electrical energies are simultaneously supplied.

### CROSS-REFERENCE TO RELATED APPLICATION

This application is a continuation application of PCT Application No. PCT/JP 2017/018546 filed on May 17, 2017, which is hereby incorporated by reference in its entirety.

### TECHNICAL FIELD

The disclosed technology relates to a control apparatus used in a treatment system including electrodes that can give a high-frequency current to a treatment target and an electrical element other than the electrode. Furthermore, the disclosed technology relates to a treatment system that incorporates the control apparatus.

### DESCRIPTION OF THE RELATED ART

In US Patent Application Pub. No. 2009/0248002A1, a treatment instrument discloses bipolar electrodes and a heater that are disposed at an end effector. In this treatment instrument, high-frequency power is supplied to the bipolar electrodes as first electrical energy. This enables a high-frequency current to flow between the bipolar electrodes and to give the high-frequency current to the treatment target. Furthermore, second electrical energy is supplied to a heater that is an electrical element. Due to this, heater heat is generated at the heater and it becomes possible to give the generated heater heat to the treatment target. Furthermore, in a treatment system in which the treatment instrument is used, output sources of the first electrical energy and the second electrical energy can simultaneously output the first electrical energy and the second electrical energy, and the high-frequency current and the heater heat can be simultaneously given to the treatment target in the treatment instrument.

In the treatment instrument as disclosed in US Patent Application Pub. No. 2009/0248002A1, a processor calculates a physical quantity relating to the first electrical energy based on a detected value detected regarding the first electrical energy and carries out output control of the first electrical energy by using the calculated physical quantity. Similarly, the processor calculates a physical quantity relating to the second electrical energy based on a detected value detected regarding the second electrical energy and carries out output control of the second electrical energy by using the calculated physical quantity. Here, in the state in which the high-frequency power with a high frequency is supplied to the electrodes as the first electrical energy and simultaneously the second electrical energy is supplied to the electrical element, there is a possibility that the detected value regarding the second electrical energy is affected by electrical noise attributed to the first electrical energy. Possibly this electrical noise affects the calculation result of the physical quantity relating to the second electrical energy. Due to the affecting of the calculated physical quantity by the noise, possibly the output control of the second electrical energy by use of the physical quantity is affected.

### BRIEF SUMMARY OF EMBODIMENTS

The disclosed technology has been made in view of the foregoing.

One aspect of the disclosed technology is directed to a control apparatus of a treatment system having respective first and second output sources that supplies respective high-frequency power defined by a first electrical energy and a second electrical energy. The control apparatus comprises a processor configured to acquire a first value based on the first electrical energy and a second value based on the second electrical energy. Based on the first value, to determine influence given to the second value by the first electrical energy. Based on the influence given to the second value by the first electrical energy and the second value based on the second electrical energy, to adjust an output instruction value to the second output source.

Another aspect of the disclosed technology is directed to a treatment system comprises a control apparatus having respective first and second output sources that supplies respective high-frequency power defined by a first electrical energy and a second electrical energy. The control apparatus includes a processor configured to acquire a first value based on the first electrical energy and a second value based on the second electrical energy. Based on the first value, the processor determines influence given to the second value by the first electrical energy. Based on the influence given to the second value by the first electrical energy and the second value based on the second electrical energy, to adjust an output instruction value to the second output source. An electrode is configured to receive the first electrical energy and an electrical component is configured to receive the second electrical energy.

A further aspect of the disclosed technology is directed to a method of operating a treatment system having respective first and second output sources that supplies respective high-frequency power defined by a first electrical energy and a second electrical energy. The method comprises acquiring a first value based on the first electrical energy and a second value based on the second electrical energy: determining influence given to the second value by the first electrical energy based on the first value and adjusting an output instruction value to the second output source based on the influence given to the second value by the first electrical energy and the second value based on the second electrical energy. The second electrical energy causes a heater to generate heat or causes an ultrasonic transducer to generate ultrasonic vibration.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a schematic diagram depicting a treatment system according to a first embodiment.

FIG. 2 is a block diagram schematically depicting a configuration that controls supply of electrical energy to a treatment instrument according to the first embodiment.

FIG. 3 is a flowchart depicting processing of calculating a physical quantity relating to second electrical energy, executed by a processor according to the first embodiment.

FIG. 4 is a schematic diagram depicting one example in which noise is superimposed on a waveform relating to the second electrical energy in the first embodiment.

FIG. 5 is a schematic diagram depicting one example of a noise correction function used when noise is superimposed as in the one example of FIG. 4.

FIG. 6 is a schematic diagram depicting one example that is different from FIG. 4 and in which noise is superimposed on a waveform relating to the second electrical energy in the first embodiment.

FIG. 7 is a schematic diagram depicting one example of a noise correction function used when noise is superimposed as in the one example of FIG. 6.

FIG. 8 is a flowchart depicting processing of calculating the physical quantity relating to the second electrical energy, executed by a processor according to a second embodiment.

FIG. 9 is a schematic diagram depicting one example of the relationship between output power from a first output source and a transform expression to the physical quantity relating to the second electrical energy according to the second embodiment.

FIG. 10 is a schematic diagram depicting one example of multiple transform functions that become options when a treatment system has the relationship between the output power and the transform expression depicted in FIG. 9.

FIG. 11 is a schematic diagram depicting one example different from FIG. 10 regarding multiple transform functions that become options when the treatment system has the relationship between the output power and the transform expression depicted in

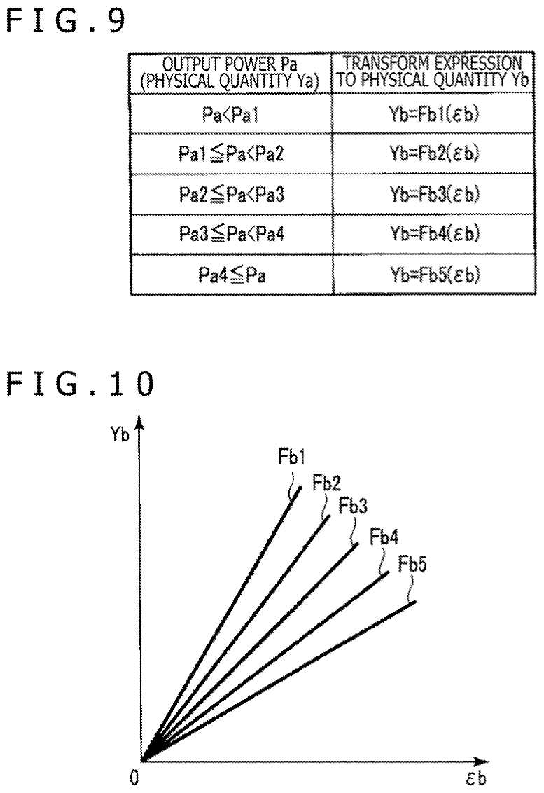

FIG. 9.

FIG. 12 is a block diagram schematically depicting a configuration that controls supply of the electrical energy to the treatment instrument according to a certain modification example.

FIG. 13 is a block diagram schematically depicting a configuration that controls supply of the electrical energy to the treatment instrument according to a certain modification example different from FIG. 12.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

An object of the disclosed technology is to provide a control apparatus that properly calculates a physical quantity relating to each of electrical energies even when electrical noise is generated in the state in which multiple electrical energies are simultaneously supplied. Furthermore, an object of the disclosed technology is to provide a treatment system integrates the control apparatus thereto.

### First Embodiment

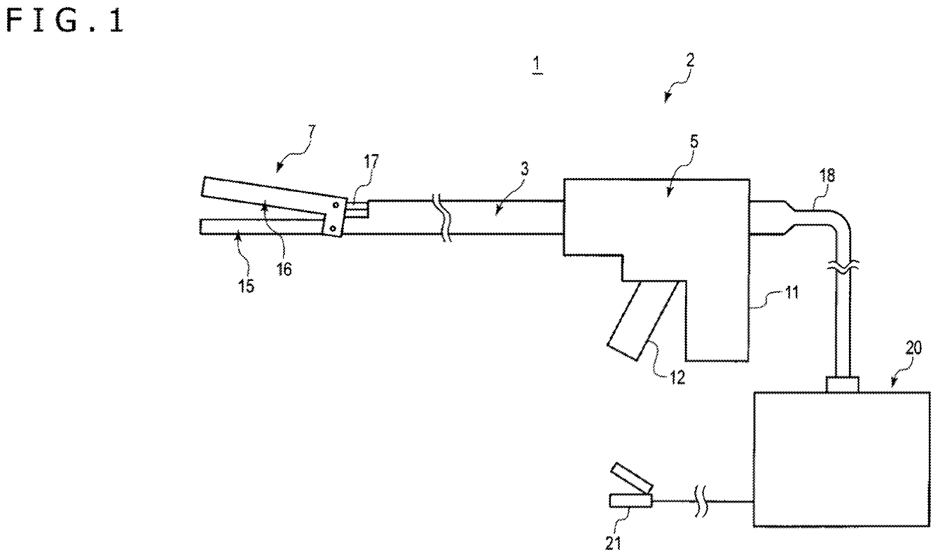

A first embodiment of the disclosed technology will be described with reference to FIG. 1 to FIG. 7. FIG. 1 is a diagram depicting a treatment system 1 of the present embodiment. As depicted in FIG. 1, the treatment system 1 includes a treatment instrument 2 and the treatment instrument 2 includes a tubular shaft 3, a housing 5, and an end effector 7. The housing 5 is joined to one side of the shaft 3 regarding the direction along the center axis of the shaft 3. Furthermore, a grip 11 is made in the housing 5 that can be held and a handle 12 is pivotally attached to the housing 5. Through the pivot of the handle 12, the handle 12 opens or closes with respect to the grip 11.

The end effector 7 is disposed at the end part on the opposite side to the side on which the housing 5 is located regarding the direction along the center axis of the shaft 3 in the shaft 3. Therefore, the shaft 3 is extended from the end effector 7 toward the housing 5. The end effector 7 includes a pair of grasping pieces, or clamp members, 15 and 16 and at least one of the grasping pieces 15 and 16 can pivot with respect to the shaft 3. Furthermore, inside or outside the shaft 3, a movable member 17 is extended along the center axis of the shaft 3. One end of the movable member 17 is joined to the handle 12 inside the housing 5 and the other end of the movable member 17 is connected to the end effector 7. By opening or closing the handle 12 with respect to the grip 11, the movable member 17 moves along the center axis of the shaft 3. Thus, at least one of the grasping pieces 15 and 16 pivots and the grasping pieces 15 and 16 open or close with respect to one another. Therefore, at the end effector 7, a treatment target such as biological tissue can be grasped between the grasping pieces 15 and 16.

One end of a cable 18 is connected to the housing 5. The other end of the cable 18 is connected to a power supply apparatus 20 that is a separate portion from the treatment instrument 2. Furthermore, an operation apparatus 21 is disposed in the treatment system 1. In the embodiment example of FIG. 1, the operation apparatus 21 is a foot switch that is a separate portion from the treatment instrument 2 and is electrically connected to the power supply apparatus 20. The power supply apparatus 20 supplies electrical energy to the treatment instrument 2 based on operation at the operation apparatus 21. In a certain embodiment example, as the operation apparatus, or operation member, 21, an operation button or the like attached to the housing 5 is disposed instead of the foot switch or in addition to the foot switch.

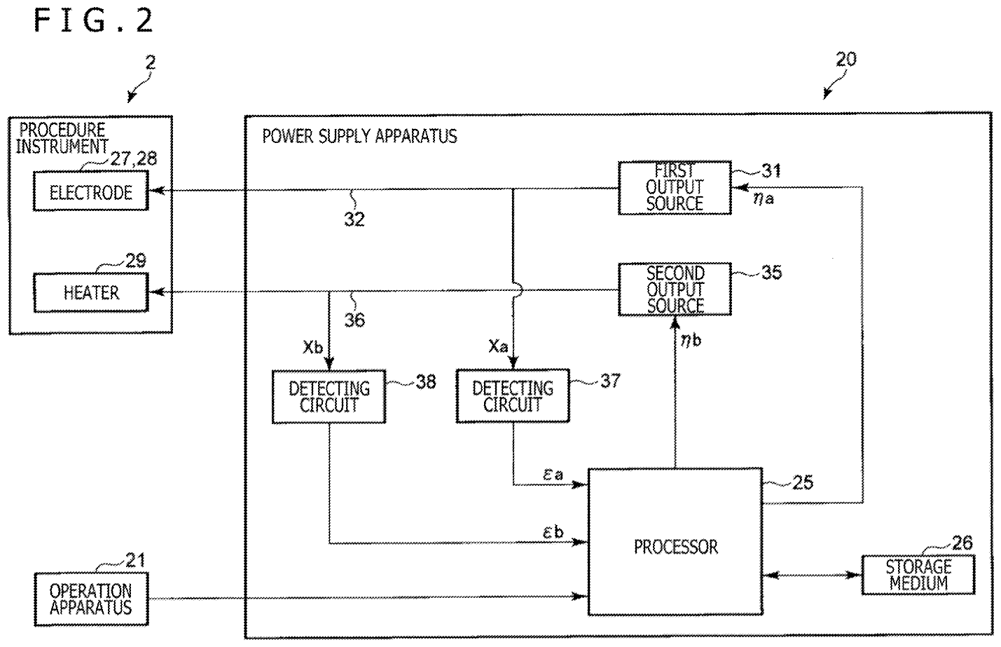

FIG. 2 is a diagram depicting a configuration that controls supply of electrical energy to the treatment instrument 2. As depicted in FIG. 2, the power supply apparatus 20 includes a processor, or controller, 25 and a storage medium 26. The processor 25 is formed of an integrated circuit or the like including a central processing unit (CPU), application specific integrated circuit (ASIC), field programmable gate array (FPGA), or the like. One processor 25 may be disposed in the power supply apparatus 20 or multiple processors 25 may be disposed in the power supply apparatus 20. In the present embodiment, the processor 25 configures at least part of the control apparatus. Processing in the processor 25 is executed in accordance with a program stored in the processor 25 or the storage medium 26. Furthermore, in the storage medium 26, processing programs used in the processor 25 and parameters used in arithmetic operation in the processor 25, functions, tables, and so forth are stored. The processor 25 detects whether or not operation has been input in the operation apparatus 21 such as a foot switch.

Furthermore, in the present embodiment, electrodes 27 and 28 are disposed at the end effector 7 of the treatment instrument 2 as bipolar electrodes. The electrode 27 is disposed on one of the grasping pieces 15 and 16 and the electrode 28 is disposed on the other of the grasping pieces 15 and 16. Furthermore, a heater 29 is disposed at the end effector 7 as an electrical element. The heater 29 is disposed on at least one of the grasping pieces 15 and 16.

The power supply apparatus 20 includes a first output source, or high-frequency power supply, 31. The first output source 31 includes waveform generator, conversion circuit, transformer, and so forth and converts power from a battery power supply, outlet power supply, or the like to high-frequency power that is first electrical energy. Then, the first output source 31 outputs the converted high-frequency power. On this occasion, for example, the high-frequency power with a frequency that is equal to or higher than 200 kHz and is preferably equal to or higher than 300 kHz and equal to or lower than 1 MHz is output. A first electrical path 32 is formed between the first output source 31 and the electrodes 27 and 28. The first electrical path 32 includes a connection path that electrically connects the first output source 31 and the electrode 27 and a connection path that electrically connects the first output source 31 and the electrode 28. In the present embodiment, the first electrical path 32 is extended to pass through the inside of the shaft 3, the inside of the housing 5, and the inside of the cable 18. The first output source 31 supplies the output high-frequency power, or first electrical energy, to the electrodes 27 and 28 through the first electrical path 32. Therefore, the first electrical path 32 forms a supply path of the first electrical energy to the electrodes 27 and 28. Due to the supply of the high-frequency power to the electrodes 27 and 28, the electrodes 27 and 28 have potentials different from one another. Thus, through the supply of the high-frequency power to the electrodes 27 and 28 in the state in which a treatment target is grasped between the grasping pieces 15 and 16, a high-frequency current flows between the electrodes 27 and 28 through the treatment target and the high-frequency power is given to the treatment target as treatment energy.

Furthermore, the power supply apparatus 20 includes a second output source, or heater power supply, 35. The second output source 35 includes conversion circuit, transformer, and so forth and converts power from a battery power supply, outlet power supply, or the like to second electrical energy. Then, the second output source 35 outputs the converted second electrical energy. On this occasion, direct-current power or alternating-current power is output as the second electrical energy. Furthermore, if alternating-current power is output as the second electrical energy, alternating-current power with a frequency lower than 200 kHz is output, for example. A second electrical path 36 is formed between the second output source 35 and the heater 29. The second electrical path 36 includes two connection paths that each electrically connect the second output source 35 and the heater 29. In the present embodiment, the second electrical path 36 is extended to pass through the inside of the shaft 3, the inside of the housing 5, and the inside of the cable 18.

Heater 29 receives electrical energy from the second output source 35 and the heat is generated in the heater 29. The heat generated by the heater 29 is transmitted as treatment energy to the treatment target via the grasping pieces 15 and/or 16.

When detecting operation input at the operation apparatus 21, the processor 25 transmits, for example, a digital signal to each of the output sources 31 and 35 as an output instruction. By the transmission of the output instruction from the processor 25 to the output sources 31 and 35, the high-frequency power is output from the first output source 31 and the second electrical energy is output from the second output source 35. Here, when an instruction value ηa of the output instruction to the first output source 31, i.e. the signal value of the digital signal to the first output source 31, changes, the state of the output from the first output source 31 changes and the supply state of the high-frequency power to the electrodes 27 and 28 changes. The processor 25 controls the output from the first output source 31 and controls the supply of the high-frequency power, or first electrical energy, to the electrodes 27 and 28 by adjusting the instruction value ηa of the output instruction. Furthermore, when an instruction value ηb of the output instruction to the second output source 35, i.e. the signal value of the digital signal to the second output source 35, changes, the state of the output from the second output source 35 changes and the supply state of the second electrical energy to the heater 29 changes. The processor 25 controls the output from the second output source 35 and controls the supply of the second electrical energy to the heater 29 by adjusting the instruction value ηb of the output instruction. The processor 25 acquires the instruction values ηa and ηb in the state in which the processor 25 is transmitting the output instructions to the output sources 31 and 35.

Moreover, detecting circuits 37 and 38 are disposed in the power supply apparatus 20. The detecting circuit, or first detecting circuit, 37 detects a measurement amount Xa relating to the first electrical energy at the first electrical path 32. The detecting circuit 37 includes at least one of a current detecting circuit and a voltage detecting circuit and the detecting circuit 37 measures, for example, at least one of an output current to the electrodes 27 and 28 and an output voltage to the electrodes 27 and 28 as the measurement amount Xa that is a detection target. Then, in the detecting circuit 37, the measurement result is converted to a digital signal and the converted digital signal is transmitted from the detecting circuit 37 to the processor 25. The processor 25 acquires the signal value of the transmitted digital signal as a detected value εa detected regarding the first electrical energy. For example, with the digital signal converted from the output current to the electrodes 27 and 28, the detected value εa becomes a value that represents information relating to the output current to the electrodes 27 and 28. Furthermore, with the digital signal converted from the output voltage to the electrodes 27 and 28, the detected value εa becomes a value that represents information relating to the output voltage to the electrodes 27 and 28.

The detecting circuit, or second detecting circuit, 38 detects a measurement amount Xb relating to the second electrical energy at the second electrical path 36. The detecting circuit 38 measures, for example, at least one of an output current to the heater 29 and an output voltage to the heater 29 as the measurement amount Xb that is a detection target. Then, in the detecting circuit 38, the measurement result is converted to a digital signal and the converted digital signal is transmitted from the detecting circuit 38 to the processor 25. The processor 25 acquires the signal value of the transmitted digital signal as a detected value εb detected regarding the second electrical energy. For example, with the digital signal converted from the output current to the heater 29, the detected value εb becomes a value that represents information relating to the output current to the heater 29. Furthermore, with the digital signal converted from the output voltage to the heater 29, the detected value εb becomes a value that represents information relating to the output voltage to the heater 29.

Here, in each of the detecting circuits 37 and 38, the digital signal to be transmitted to the processor 25 is generated by using a known circuit configuration and so forth. In a certain embodiment example, alternating-current power is output from the second output source 35 to the heater 29 and an analog signal that represents the waveform of an alternating-current voltage corresponding to the waveform of an output voltage to the heater 29 is input to the detecting circuit 38 as the measurement amount Xb. In this case, in the detecting circuit 38, voltage dividing, half-wave rectification, alternating current (AC)/direct current (DC) conversion, and analog-to-digital (A/D) conversion are sequentially carried out for the input waveform and the digital signal to be transmitted to the processor 25 is generated. In another certain embodiment example, half-wave rectification and AC/DC conversion are not carried out and a waveform resulting from voltage dividing is subjected to A/D conversion. Furthermore, in another certain embodiment example, direct-current power is output to the heater 29 and an analog signal that represents a direct-current voltage corresponding to an output voltage to the heater 29 is input to the detecting circuit 38 as the measurement amount Xb. In this case, in the detecting circuit 38, voltage dividing and A/D conversion are sequentially carried out for the input direct-current voltage and the digital signal is generated.

Moreover, in a certain embodiment example, an analog signal that represents the waveform of an alternating-current current corresponding to the waveform of an output current to the heater 29 is input to the detecting circuit 38 as the measurement amount Xb. In this case, in the detecting circuit 38, current/voltage conversion, half-wave rectification, AC/DC conversion, and A/D conversion are sequentially carried out for the input waveform and the digital signal is generated. In another certain embodiment example, half-wave rectification and AC/DC conversion are not carried out and a waveform resulting from current/voltage conversion is subjected to A/D conversion. Furthermore, in another certain embodiment example, an analog signal that represents a direct-current current corresponding to an output current to the heater 29 is input to the detecting circuit 38 as the measurement amount Xb. In this case, in the detecting circuit 38, current/voltage conversion and A/D conversion are sequentially carried out for the input direct-current current and the digital signal is generated.

The processor 25 calculates a physical quantity Ya relating to the first electrical energy by using the detected value εa regarding the acquired first electrical energy. On this occasion, the processor 25 calculates the physical quantity Ya by using a transform function, or first transform function, Fa stored in the storage medium 26, for example. Here, as the physical quantity Ya, at least one of output current, output voltage, output power, and output frequency to the electrodes 27 and 28, the impedance between the electrodes 27 and 28, or impedance of the treatment target, and the phase difference between the output current and the output voltage is calculated. In a certain embodiment example, at least one of output current and output voltage to the electrodes 27 and 28 is input to the detecting circuit 37 as the measurement amount Xa and is calculated as the physical quantity Ya by the processor 25.

Furthermore, the processor 25 calculates a physical quantity Yb relating to the second electrical energy by using the detected value εa regarding the acquired first electrical energy and the detected value εb regarding the second electrical energy. On this occasion, the processor 25 calculates the physical quantity Yb by using a transform function, or second transform function, Fb and a noise correction function fb stored in the storage medium 26, for example. Here, as the physical quantity Yb, at least one of output current, output voltage, output power, and output frequency to the heater 29, the resistance value of the heater 29, the temperature of the heater 29 calculated based on the resistance value of the heater 29, and the phase difference between the output current and the output voltage is calculated. In a certain embodiment example, at least one of output current and output voltage to the heater 29 is input to the detecting circuit 38 as the measurement amount Xb and is calculated as the physical quantity Yb by the processor 25. Details of the calculation of the physical quantities Ya and Yb will be described hereinafter.

The processor 25 adjusts the instruction value ηa of the output instruction to the first output source 31 and controls the output of the high-frequency power to the electrodes 27 and 28 based on the calculated physical quantity Ya. In a certain embodiment example, the processor 25 calculates the impedance between the electrodes 27 and 28 as the physical quantity Ya and controls the output to the electrodes 27 and 28 to control the supply of the high-frequency power to the electrodes 27 and 28 based on the calculated impedance. Furthermore, in a certain embodiment example, the processor controls the output to the electrodes 27 and 28 based on the physical quantity Yb relating to the calculated second electrical energy in addition to the physical quantity Ya.

Moreover, the processor 25 adjusts the instruction value ηb of the output instruction to the second output source 35 and controls the output of the second electrical energy to the heater 29 based on the calculated physical quantity Yb. In a certain embodiment example, the processor 25 calculates the temperature of the heater 29 as the physical quantity Yb and controls the output to the heater 29 to control the supply of the second electrical energy to the heater 29 based on the calculated temperature. Furthermore, in a certain embodiment example, the processor 25 controls the output to the heater 29 based on the physical quantity Ya relating to the calculated first electrical energy in addition to the physical quantity Yb.

Next, operation and effect of the processor 25 and the treatment system 1 in the present embodiment will be described. When treatment is carried out by using the treatment system 1, the end effector 7 is inserted into a body cavity such as an abdominal cavity. Then, a treatment target is disposed between the grasping pieces 15 and 16 and an operator closes the handle 12 with respect to the grip 11 to close the grasping pieces and 16 with respect to each other. Thus, the treatment target is grasped between the grasping pieces 15 and 16. Through input of operation with the operation apparatus 21 by the operator in the state in which the treatment target is grasped, the processor 25 transmits an output instruction to each of the output sources 31 and 35. Thereby, high-frequency power, or first electrical energy, is output from the first output source 31 to the electrodes 27 and 28 and the second electrical energy is output from the second output source 35 to the heater, or electrical element, 29. Therefore, simultaneously with the flowing of a high-frequency current between the electrodes 27 and 28 through the treatment target, heater heat is given to the treatment target.

In the state in which the first electrical energy, or high-frequency power, and the second electrical energy are simultaneously output, an analog signal that represents the measurement amount Xa relating to the first electrical energy is input to the detecting circuit 37 and an analog signal that represents the measurement amount Xb relating to the second electrical energy is input to the detecting circuit 38. The input analog signal is converted to the digital signal in the detecting circuit 37 as described hereinbefore and the processor 25 acquires the signal value of the digital signal as the detected value εa detected regarding the first electrical energy. In the present embodiment, the detected value εa is used as a first electrical value in calculation of each of the physical quantities Ya and Yb to be described hereinafter. Furthermore, the input analog signal is converted to the digital signal in the detecting circuit 38 as described hereinbefore and the processor acquires the signal value of the digital signal as the detected value εb detected regarding the second electrical energy. In the present embodiment, the detected value εb is used as a second electrical value in calculation of the physical quantity Yb to be described hereinafter.

The processor 25 calculates the physical quantity Ya relating to the first electrical energy by using the acquired detected value, or first electrical value, εa and the transform function Fa stored in the storage medium 26 or the like. In the present embodiment, the processor 25 calculates the physical quantity Ya by substituting the detected value εb into the transform function Fa. Then, the processor 25 adjusts the instruction value ηa of the output instruction to the first output source 31 and controls the output of the high-frequency power, or first electrical energy, to the electrodes 27 and 28 at least based on the calculated physical quantity Ya.

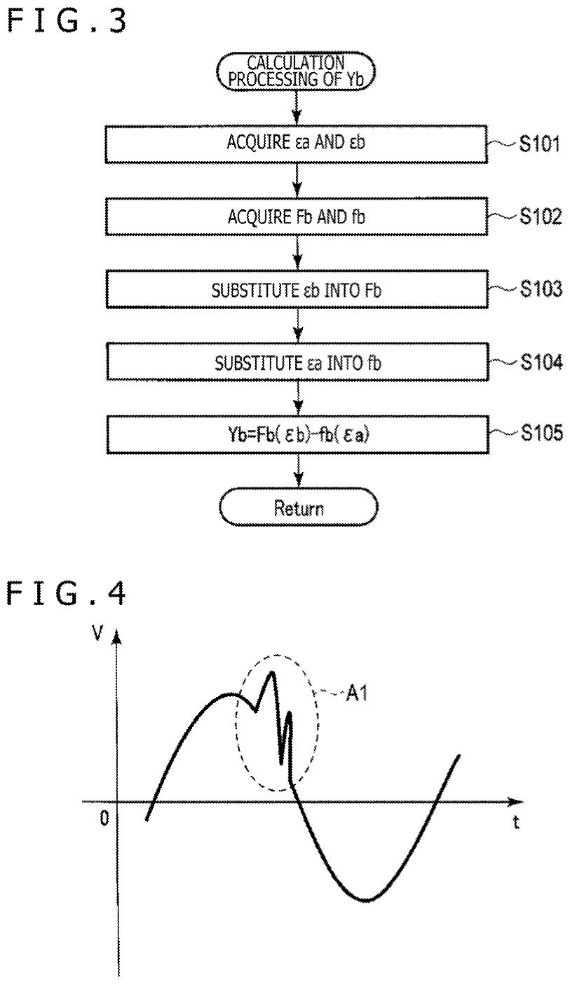

FIG. 3 is a flowchart depicting processing of calculating the physical quantity Yb relating to the second electrical energy, executed by the processor 25. As depicted in FIG. 3, in the calculation of the physical quantity Yb, the processor 25 acquires the detected value, or first electrical value, εa that is the signal value of the digital signal from the detecting circuit 37 and the detected value, or second electrical value, εb that is the signal value of the digital signal from the detecting circuit 38 in S101. Furthermore, the processor 25 acquires the transform function, or second transform function, Fb and the noise correction function fb stored in the storage medium 26 or the like in S102. Then, the processor 25 substitutes the detected value εb into the transform function Fb to carry out arithmetic operation in S103, and substitutes the detected value εa into the noise correction function fb to carry out arithmetic operation in S104. Then, the processor 25 calculates the physical quantity Yb by subtracting the arithmetic operation result (calculated value fb(εa)) using the noise correction function fb from the arithmetic operation result (calculated value Fb(εb)) using the transform function Fb in S105. The processor 25 adjusts the instruction value ηb of the output instruction to the second output source 35 and controls the output of the second electrical energy to the heater 29 at least based on the calculated physical quantity Yb.

Here, in the present embodiment, the second electrical energy is direct-current power or alternating-current power with a low frequency compared with the high-frequency power that is the first electrical energy. For this reason, for example, in the process of converting the analog signal input to the detecting circuit 38 to the digital signal to be transmitted to the processor 25, possibly electrical noise attributed to the first electrical energy is superimposed on any waveform and/or direct-current voltage or the like.

FIG. 4 depicts one example in which noise is superimposed on a waveform relating to the second electrical energy. In the one example of FIG. 4, the noise is superimposed on a voltage waveform resulting from voltage dividing of the analog signal input to the detecting circuit 38. In FIG. 4, a time t is represented on the abscissa axis and a voltage V is represented on the ordinate axis. In the one example of FIG. 4, due to the superposition of the noise, a deviation region A1 that deviates in the positive direction relative to the waveform on which the noise is not superimposed is generated. In this case, the signal value of the digital signal to be transmitted to the processor 25, i.e. the detected value εb, increases compared with the case in which noise is not superimposed. Due to the increase in the detected value εb, the calculated value Fb(εb) figured out by using the transform function Fb increases compared with the case in which noise is not superimposed. Furthermore, if noise is superimposed, when the output from the first output source 31 is higher, the deviation from the waveform on which the noise is not superimposed is larger in the deviation region A1 attributed to the noise. For this reason, if noise is superimposed, when the output from the first output source 31 is higher, the difference in the detected value εb relative to the case in which noise is not superimposed is larger and the difference in the calculated value Fb(εb) relative to the case in which noise is not superimposed is larger.

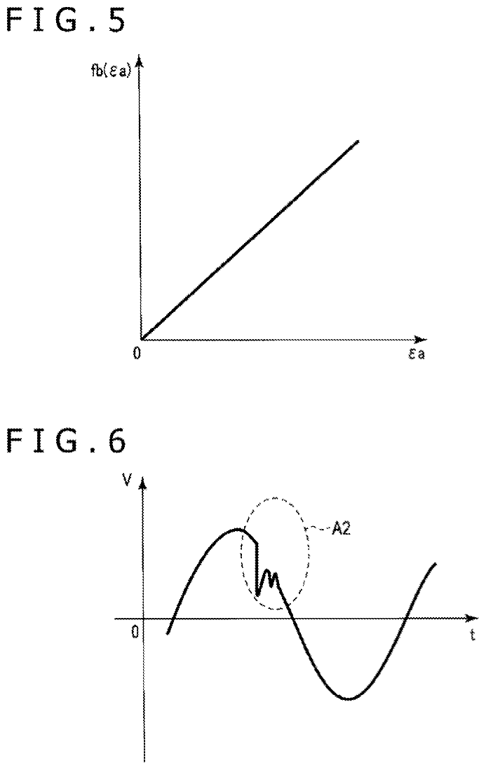

FIG. 5 is a diagram depicting one example of the noise correction function fb used when noise is superimposed as in the one example of FIG. 4. In FIG. 5, the detected value εa detected regarding the first electrical energy is represented on the abscissa axis and the calculated value fb(εa) figured out by using the noise correction function fb is represented on the ordinate axis. In the one example of FIG. 5, the calculated value fb(εa) becomes zero or a positive value whatever magnitude the detected value εa has. Furthermore, the calculated value fb(εa) is larger when the detected value εa is larger. Here, when the output from the first output source 31 is higher, the measurement amount Xa input to the detecting circuit 37 is larger and the detected value εa is larger. Moreover, as described hereinbefore, when the output from the first output source 31 is higher, the influence of the noise attributed to the first electrical energy, or high-frequency power, on the detected value εb is larger and the difference in the calculated value Fb(εb) relative to the case in which noise is not superimposed is larger. Therefore, when the influence of the noise attributed to the first electrical energy on the detected value εb is larger, the difference in the calculated value Fb(εb) relative to the case in which noise is not superimposed is larger and the calculated value fb(εa) is larger.

Furthermore, in the one example depicted in FIG. 4 and FIG. 5, the physical quantity Ya relating to the second electrical energy is calculated by subtracting the calculated value fb(εa) from the calculated value Fb(εb). Thus, even when the detected value εb and the calculated value Fb(εb) increase compared with the case in which noise is not superimposed due to the influence of noise, through the subtraction of the calculated value fb(εa) from the calculated value Fb(εb), the value corresponding to the increase in the calculated value Fb(εb) due to the influence of the noise is properly corrected by arithmetic operation using the noise correction function fb. Furthermore, the calculated value fb(εa) is larger when the influence of the noise on the detected value εb is larger, i.e. when the calculated value Fb(εb) is larger. For this reason, a proper value made to correspond to the influence of the noise on the detected value εb is figured out as the calculated value fb(εa) subtracted from the calculated value Fb(εb). Because the influence of the noise is corrected by using the calculated value fb(εa) of the proper value made to correspond to the influence of the noise on the detected value εb, the proper physical quantity Yb resulting from the correction of the influence of the noise is calculated.

FIG. 6 depicts another example in which noise is superimposed on a waveform relating to the second electrical energy. Also in the one example of FIG. 6, the noise is superimposed on a voltage waveform resulting from voltage dividing of the analog signal input to the detecting circuit 38. In FIG. 6, a time t is represented on the abscissa axis and a voltage V is represented on the ordinate axis. In the one example of FIG. 6, due to the superposition of the noise, a deviation region A2 that deviates in the negative direction relative to the waveform on which the noise is not superimposed is generated. In this case, the detected value εb decreases compared with the case in which noise is not superimposed and the calculated value Fb(εb) figured out by using the transform function Fb decreases compared with the case in which noise is not superimposed. Furthermore, if noise is superimposed, when the output from the first output source 31 is higher, the deviation from the waveform on which the noise is not superimposed is larger in the deviation region A2 attributed to the noise. For this reason, if noise is superimposed, when the output from the first output source 31 is higher, the difference in the detected value εb relative to the case in which noise is not superimposed is larger and the difference in the calculated value Fb(εb) relative to the case in which noise is not superimposed is larger.

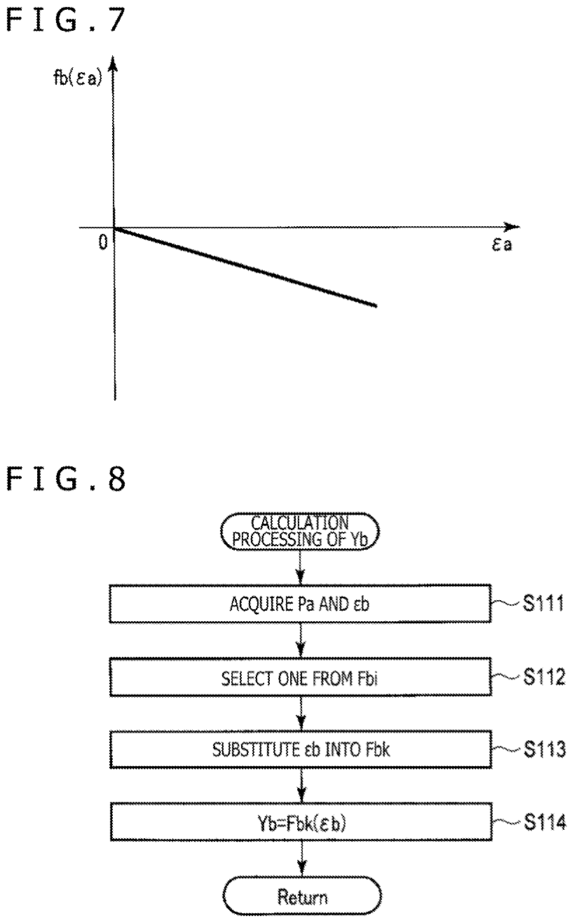

FIG. 7 is a diagram depicting one example of the noise correction function fb used when noise is superimposed as in the one example of FIG. 6. In FIG. 7, the detected value εa is represented on the abscissa axis and the calculated value fb(εa) figured out by using the noise correction function fb is represented on the ordinate axis. In the one example of FIG. 7, the calculated value fb(εa) becomes zero or a negative value whatever magnitude the detected value εa has. Furthermore, the calculated value fb(εa) is smaller when the detected value εa is larger. Therefore, when the influence of the noise attributed to the first electrical energy on the detected value εb is larger, the difference in the calculated value Fb(εb) relative to the case in which noise is not superimposed is larger and the calculated value fb(εa) is smaller.

Furthermore, in the one example depicted in FIG. 6 and FIG. 7, the physical quantity Ya relating to the second electrical energy is calculated by subtracting the calculated value fb(εa) that is zero or a negative value from the calculated value Fb(εb). Thus, even when the detected value εb and the calculated value Fb(εb) decrease compared with the case in which noise is not superimposed due to the influence of noise, through the subtraction of the calculated value fb(εa) from the calculated value Fb(εb), the value corresponding to the decrease in the calculated value Fb(εb) due to the influence of the noise is properly corrected by arithmetic operation using the noise correction function fb. Furthermore, the calculated value fb(εa) is smaller and the absolute value of the calculated value fb(εa) is larger when the influence of the noise on the detected value εb is larger, i.e. when the calculated value Fb(εb) is smaller. For this reason, a proper value made to correspond to the influence of the noise on the detected value εb is figured out as the calculated value fb(εa) subtracted from the calculated value Fb(εb). Because the influence of the noise is corrected by using the calculated value fb(εa) of the proper value made to correspond to the influence of the noise on the detected value εb, the proper physical quantity Yb resulting from the correction of the influence of the noise is calculated.

As described hereinbefore, in the present embodiment, the influence of electrical noise given to the detected value, or second electrical value, εb by the first electrical energy is determined based on the detected value, or first electrical value, εa. Then, a proper value made to correspond to the determination result of the influence of the noise is figured out as the calculated value fb(εa) by using the detected value εa and the noise correction function fb. Then, the physical quantity Yb is calculated based on the calculated value fb(εa) made to correspond to the determination result of the influence of the noise in addition to the detected value εb. For this reason, in the present embodiment, even when electrical noise is generated and the detected value εb is affected by the noise, the physical quantity Yb relating to the calculated second electrical energy becomes the proper value resulting from the correction of the influence of the noise. Through execution of output control of the second electrical energy with use of the physical quantity Yb of the proper value, supply of the second electrical energy to the heater, or electrical element, 29 is properly controlled and the performance of treatment using heater heat is improved. Furthermore, if output control of the first electrical energy is also carried out by using the physical quantity Yb, due to the calculation of the physical quantity Yb of the proper value, supply of high-frequency power to the electrodes 27 and 28 is properly controlled and the performance of treatment using a high-frequency current is improved.

Moreover, in the present embodiment, the influence of noise is corrected in the process of calculating the physical quantity Yb by processing in the processor 25. Therefore, the influence of electrical noise is corrected in an algorithm of calculating the physical quantity Yb by the processor 25, i.e. in software. Accordingly, the physical quantity Yb resulting from the correction of the influence of the noise is calculated without adding hardware (parts).

### Modification Example of First Embodiment

In the first embodiment, the first electrical value substituted into the noise correction function fb is the detected value εa. However, the configuration is not limited thereto. In a certain modification example, the noise correction function fb different from the first embodiment is used and the processor 25 substitutes the instruction value ηa of the output instruction to the first output source 31, instead of the detected value εa, into the noise correction function fb to carry out arithmetic operation in the processing of S104. In this case, the processor 25 acquires the instruction value ηa instead of acquiring the detected value εa in the processing of S101. Therefore, in the present modification example, the processor 25 determines the influence of noise attributed to the first electrical energy on the detected value εb regarding the second electrical energy based on the instruction value ηa used as the first electrical value instead of the detected value εa. Furthermore, in the processing of S105, the processor 25 calculates the physical quantity Yb by subtracting the arithmetic operation result obtained by substituting the instruction value ηa into the noise correction function fb from the arithmetic operation result using the transform function Fb.

Here, when the instruction value ηa changes, the state of the output from the first output source 31 changes and the measurement amount Xa that is the output current and/or output voltage from the first output source 31 changes. Due to the change in the measurement amount Xa, the detected value εa regarding the first electrical energy also changes. Therefore, when the instruction value ηa changes, the detected value εa changes corresponding to the instruction value ηa. For this reason, also when the instruction value ηa is substituted into the noise correction function fb and arithmetic operation is carried out, i.e. also when noise is corrected by using the instruction value ηa as the first electrical value instead of the detected value εa, the influence of noise attributed to the first electrical energy on the detected value εb is properly corrected and the proper physical quantity Yb resulting from the correction of the influence of the noise is calculated similarly to the first embodiment.

Furthermore, in a certain modification example in which any of the output current, the output voltage, and output power Pa from the first output source 31 is calculated as the physical quantity Ya relating to the first electrical energy, the processor 25 calculates the physical quantity Yb by using the physical quantity Ya calculated by using the transform function Fa and the detected value εa as the first electrical value instead of the detected value εa. In this case, the noise correction function fb different from the embodiment described hereinbefore and so forth is used and any of the output current, the output voltage, and the output power Pa from the first output source 31 calculated as the physical quantity Ya is substituted into the noise correction function fb to carry out arithmetic operation. When the instruction value ηa changes, the state of the output from the first output source 31 changes and thus the output current, the output voltage, and the output power Pa from the first output source 31 change. Furthermore, as described hereinbefore, the detected value εa changes through change in the state of the output from the first output source 31. Therefore, the detected value εa changes corresponding to change in any of the output current, the output voltage, and the output power Pa from the first output source 31. Accordingly, also when noise is corrected by using any of the output current, the output voltage, and the output power Pa from the first output source 31 as the first electrical value instead of the detected value εa, the influence of noise attributed to the first electrical energy on the detected value εb is properly corrected and the proper physical quantity Yb resulting from the correction of the influence of the noise is calculated similarly to the first embodiment.

Furthermore, in the embodiment described hereinbefore and so forth, the physical quantity Ya relating to the first electrical energy is calculated by substituting the detected value εa into the transform function Fa. However, the configuration is not limited thereto. In a certain modification example, the physical quantity Ya may be calculated by using a noise correction function fa and the detected value εb in addition to the transform function Fa and the detected value εa. In this case, for example, the processor 25 figures out a calculated value Fa(εa) obtained by substituting the detected value εa into the transform function Fa and figures out a calculated value fa(εb) obtained by substituting the detected value εb into the noise correction function fa. Then, the processor 25 calculates the value obtained by subtracting the calculated value fa(εb) from the calculated value Fa(εa) as the physical quantity Ya. In the present modification example, the influence of electrical noise attributed to the second electrical energy on the detected value εa is corrected by subtracting the calculated value fa(εb).

In a certain modification example, the noise correction function fa different from the case of substituting the detected value εb may be used and the instruction value ηb to the second output source 35 may be substituted into the noise correction function fa instead of the detected value εb. Furthermore, if the first electrical energy is high-frequency power and the second electrical energy is direct-current power or alternating-current power with a lower frequency than the first electrical energy, or high-frequency power, as in the first embodiment, the detected value εa regarding the first electrical energy is hardly affected by electrical noise attributed to the second electrical energy. Therefore, even when the noise correction function fa is not used for the calculation of the physical quantity Ya, the physical quantity Ya becomes a proper value as in the first embodiment and so forth.

Furthermore, in a certain modification example, the processor 25 substitutes the second electrical value such as the detected value εb into a transform table instead of the transform function Fb to carry out arithmetic operation. In addition, the processor 25 substitutes the first electrical value such as the detected value εa or the instruction value ηa into a noise correction table instead of the noise correction function fb to carry out arithmetic operation. In this case, the processor 25 calculates the physical quantity Yb relating to the second electrical energy by subtracting the arithmetic operation result using the noise correction table from the arithmetic operation result using the transform table.

Moreover, in a certain modification example, a temperature sensor that detects the temperature of the heater 29 is set and an output from the temperature sensor is input to the detecting circuit 38. In this case, the detecting circuit 38 measures the temperature of the heater 29 as the measurement amount Xb relating to the second electrical energy. Furthermore, the output from the temperature sensor is converted to a digital signal in the detecting circuit 38 and the converted digital signal is transmitted to the processor 25. In this case, with the digital signal converted from the output of the temperature sensor, the detected value εb becomes a value that represents information relating to the temperature of the heater 29.

### Second Embodiment

Next, a second embodiment of the disclosed technology will be described with reference to FIG. 8 to FIG. 11. The second embodiment is what is obtained by modifying processing in the first embodiment as follows. The same part as the first embodiment is given the same numeral reference and description thereof is omitted.

FIG. 8 is a flowchart depicting processing of calculating the physical quantity Yb relating to the second electrical energy, executed by the processor 25 of the present embodiment. As depicted in FIG. 8, in the present embodiment, in the calculation of the physical quantity Yb, the processor 25 acquires the output power, or first electrical value, Pa from the first output source 31 calculated as the physical quantity Ya and the detected value, or second electrical value, εb regarding the second electrical energy in S111. Here, the output power Pa that is the physical quantity Ya is calculated by substituting the detected value εa into the transform function Fa as described hereinbefore in the first embodiment. Furthermore, in the present embodiment, multiple transform functions Fbi (i=1, 2, . . . , n) different from each other are stored in the storage medium 26 or the like, and five transform functions Fbi (i=1, 2, 3, 4, 5) are stored in a certain embodiment example, for example. The processor 25 selects one transform function Fbk (k is corresponding one of 1 to 5) corresponding to the output power Pa from the multiple transform functions Fbi based on the output power Pa acquired as the first electrical value relating to the first electrical energy in S112. Then, the processor 25 substitutes the detected value εb into the selected transform function Fbk to carry out arithmetic operation in S113, and figures out the arithmetic operation result (calculated value Fbk(εb)) using the transform function Fbk as the physical quantity Yb in S114.

FIG. 9 depicts one example of the relationship between the output power Pa from the first output source 31 and a transform expression to the physical quantity Yb, i.e. the relationship between the output power Pa that is the first electrical value and the transform function Fbk selected in the processing of S112. In the one example depicted in FIG. 9, the transform function Fb1 is selected if the calculated output power Pa is lower than a reference value Pa1, and the transform function Fb2 is selected if the output power Pa is equal to or higher than the reference value Pa1 and lower than a reference value Pa2. Furthermore, the transform function Fb3 is selected if the output power Pa is equal to or higher than the reference value Pa2 and lower than a reference value Pa3, and the transform function Fb4 is selected if the output power Pa is equal to or higher than the reference value Pa3 and lower than a reference value Pa4. In addition, the transform function Fb5 is selected if the output power Pa is equal to or higher than the reference value Pa4. Then, in all cases, the detected value εb is substituted into the selected transform function Fbk to calculate the physical quantity Yb.

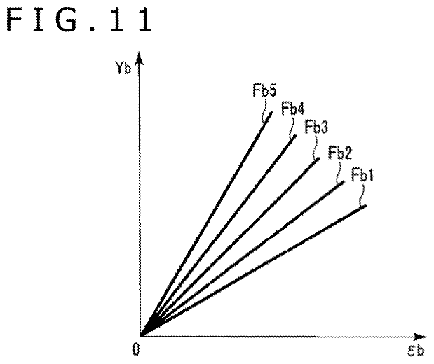

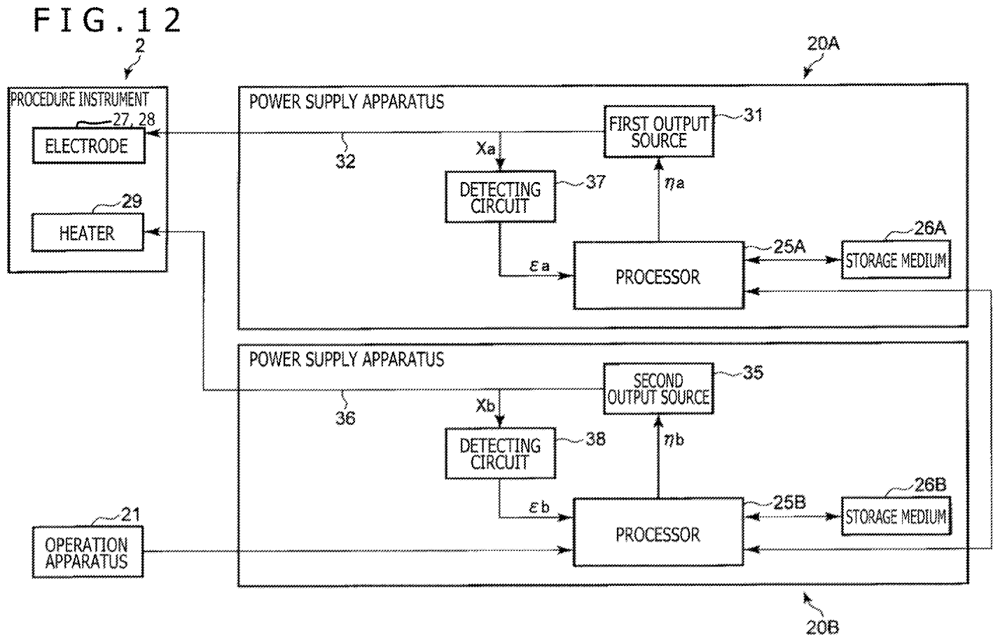

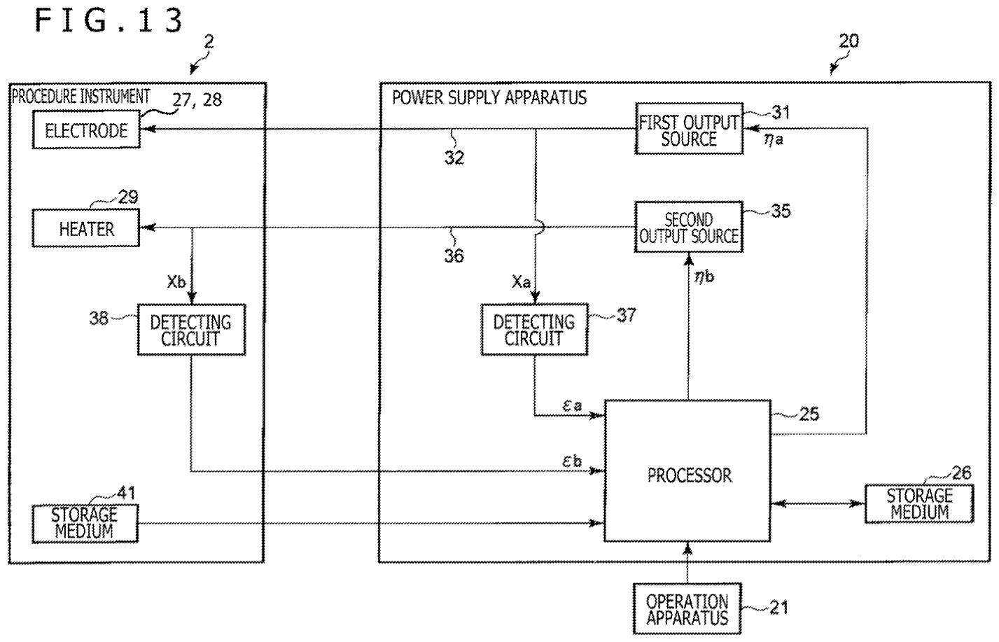

FIG. 10 depicts one example of the multiple transform functions Fbi that become options when the detected value εb regarding the second electrical energy is transformed to the physical quantity Yb relating to the second electrical energy, and FIG. 11 depicts one example different from FIG. 10 regarding the multiple transform functions Fbi that become options. In each of FIG. 10 and FIG. 11, the detected value εb is represented on the abscissa axis and the physical quantity Yb is represented on the ordinate axis. In each of FIG. 10 and FIG. 11, with any of the transform functions Fbi, the physical quantity Yb becomes equal to or larger than zero as long as the detected value εb is equal to or larger than zero. Furthermore, in each of FIG. 10 and FIG. 11, with any of the transform functions Fbi, the physical quantity Yb also increases when the detected value εb increases. However, in the one example of each of FIG. 10 and FIG. 11, the transform functions Fbi are different from each other in an increase rate β of the physical quantity Yb with respect to increase in the detected value εb. In the one example of FIG. 10, the transform functions Fb1, Fb2, Fb3, Fb4, and Fb5 are set in decreasing order of the increase rate β. Therefore, in the case of selecting the transform function Fbk used for arithmetic operation from the transform functions Fbi as in the one example of FIG. 9, the transform function Fbk with the high increase rate (slope) (3, or, for example, Fb1, is selected when the output power Pa, which is the first electrical value, is low, and the transform function Fbk with the low increase rate β, or, for example, Fb5, is selected when the output power Pa is high. On the other hand, in the one example of FIG. 11, the transform functions Fb1, Fb2, Fb3, Fb4, and Fb5 are set in increasing order of the increase rate β. Therefore, in the case of selecting the transform function Fbk used for arithmetic operation from the transform functions Fbi as in the one example of FIG. 9, the transform function Fbk with the low increase rate (slope) β, or, for example, Fb1, is selected when the output power Pa, which is the first electrical value, is low, and the transform function Fbk with the high increase rate β, or, for example, Fb5, is selected when the output power Pa is high.

For example, if the deviation region A1 that deviates in the positive direction relative to the waveform on which noise is not superimposed is generated as in the one example of FIG. 4, the detected value εb increases compared with the case in which noise is not superimposed. Furthermore, in the one example of FIG. 4, when the output from the first output source 31 is higher, i.e. when the output power Pa is higher, the difference in the detected value εb relative to the case in which noise is not superimposed is larger and the detected value εb is larger. In this case, in a certain embodiment example, the transform function Fbk is selected as depicted in the one example of FIG. 9 and FIG. 10. Thus, when the output power Pa is low and noise is hardly superimposed, the transform function Fbk with the high increase rate β, or, for example, Fb1, is used for calculation of the physical quantity Yb. On the other hand, when the output power Pa is high and the deviation in the deviation region A1 attributed to the noise is large, the transform function Fbk with the low increase rate β, or, for example, Fb5, is used for calculation of the physical quantity Yb. That is, when the influence of the noise attributed to the first electrical energy on the detected value εb is larger, the transform function Fbk with the lower increase rate β is selected. Due to this, the transform function Fbk with the proper increase rate made to correspond to the influence of the noise on the detected value εb is used for calculation of the physical quantity Yb and the proper physical quantity Yb resulting from the correction of the influence of the noise is calculated.

Moreover, for example, if the deviation region A2 that deviates in the negative direction relative to the waveform on which noise is not superimposed is generated as in the one example of FIG. 6, the detected value εb decreases compared with the case in which noise is not superimposed. Furthermore, in the one example of FIG. 6, when the output from the first output source 31 is higher, i.e. when the output power Pa is higher, the difference in the detected value εb relative to the case in which noise is not superimposed is larger and the detected value εb is smaller. In this case, in a certain embodiment example, the transform function Fbk is selected as depicted in the one example of FIG. 9 and FIG. 11. Thus, when the output power Pa is low and noise is hardly superimposed, the transform function Fbk with the low increase rate β, or, for example, Fb1, is used for calculation of the physical quantity Yb. On the other hand, when the output power Pa is high and the deviation in the deviation region A2 attributed to the noise is large, the transform function Fbk with the low increase rate β, or, for example, Fb5, is used for calculation of the physical quantity Yb. That is, when the influence of the noise attributed to the first electrical energy on the detected value εb is larger, the transform function Fbk with the higher increase rate β is selected. Due to this, the transform function Fbk with the proper increase rate β made to correspond to the influence of the noise on the detected value εb is used for calculation of the physical quantity Yb and the proper physical quantity Yb resulting from the correction of the influence of the noise is calculated.

As described hereinbefore, in the present embodiment, the influence of electrical noise given to the detected value, or second electrical value, εb by the first electrical energy is determined based on the output power, or first electrical value, Pa. Then, based on the determination result of the influence of the noise determined by using the output voltage Pa, the transform function Fbk with the proper increase rate β made to correspond to the determination result of the influence of the noise is selected from the multiple transform functions Fbi. That is, one transform function Fbi corresponding to the determination result of the influence of the noise is properly selected from the multiple transform functions Fbi and the properly-selected transform function Fbk is used for calculation of the physical quantity Yb. For this reason, also in the present embodiment, even when electrical noise is generated and the detected value εb is affected by the noise, the physical quantity Yb relating to the calculated second electrical energy becomes the proper value resulting from the correction of the influence of the noise. Therefore, also in the present embodiment, the same operation and effects as the embodiment described hereinbefore and so forth are achieved.

### Modification Example of Second Embodiment

In the second embodiment, based on the output power Pa, the transform function Fbk corresponding to the output power Pa is selected from the multiple transform functions Fbi. However, the configuration is not limited thereto. In a certain modification example, the transform function Fbk used for calculation of the physical quantity Yb is selected based on the instruction value ηa or the detected value εa instead of the output power Pa. Furthermore, in a certain modification example in which either of output current and output voltage from the first output source 31 is calculated as the physical quantity Ya relating to the first electrical energy, the processor 25 selects the transform function Fbk used for calculation of the physical quantity Yb based on either of output current and output voltage calculated as the physical quantity Ya. As described hereinbefore, when the instruction value ηa changes, the output current, the output voltage, and the output power Pa from the first output source 31 change corresponding to the instruction value ηa and the detected value εa changes corresponding to the instruction value ηa. For this reason, the proper physical quantity Yb resulting from the correction of the influence of noise is calculated also when the transform function Fbk is selected based on the instruction value ηa, the detected value εa, or either of output current and output voltage from the first output source 31 instead of the output power Pa.

Moreover, in a certain modification example, in the storage medium 26 or the like, multiple transform tables are stored instead of multiple transform functions. In this case, the processor 25 selects one transform table corresponding to the first electrical value from the multiple transform tables based on the first electrical value such as the detected value εa or the instruction value ηa. Then, the processor 25 calculates the physical quantity Yb relating to the second electrical energy by substituting the second electrical value such as the detected value εb into the selected transform table.

### Other Modification Examples

In the embodiments described hereinbefore and so forth, the output sources 31 and 35 are disposed in one power supply apparatus 20. However, the configuration is not limited thereto. In a certain modification example depicted in FIG. 12, two power supply apparatuses 20A and 20B are disposed in the treatment system 1. In the present modification example, the first output source 31 and the detecting circuit 37 are disposed in the power supply apparatus 20A and the second output source 35 and the detecting circuit 38 are disposed in the power supply apparatus 20B. Furthermore, a processor 25A and a storage medium 26A are disposed in the power supply apparatus 20A and a processor 25B and a storage medium 26B are disposed in the power supply apparatus 20B. The processors 25A and 25B can transmit information to each other in a wired or wireless manner. Furthermore, in the present modification example, at least one of the processors 25A and 25B configures at least part of a control apparatus that executes the processing described hereinbefore.

In the present modification example, the processor 25A transmits an output instruction to the first output source 31 and acquires the detected value εa relating to the first electrical energy, or high-frequency power, based on a digital signal from the detecting circuit 37. Furthermore, the processor 25B transmits an output instruction to the second output source 35 and acquires the detected value εb relating to the second electrical energy based on a digital signal from the detecting circuit 38.

Moreover, in another certain modification example depicted in FIG. 13, the detecting circuit, or second detecting circuit, 38 that detects the measurement amount Xb relating to the second electrical energy is disposed in the treatment instrument 2. Also in the present modification example, an analog signal that represents the measurement amount Xb of the output current and/or output voltage or the like from the second output source 35 is input to the detecting circuit 38 and the detecting circuit 38 converts the input analog signal to a digital signal in the manner described hereinbefore to transmit the digital signal to the processor 25 of the power supply apparatus 20. Also in the present modification example, the processor 25 acquires the signal value of the digital signal from the detecting circuit 38 as the detected value εb relating to the second electrical energy.

Furthermore, in the present modification example, a storage medium 41 is disposed also in the treatment instrument 2. The transform function Fb and the noise correction function fb described hereinbefore and so forth are stored in the storage medium 41. For this reason, for each treatment instrument 2, the storage medium 41 can be caused to store the transform function Fb and the noise correction function fb corresponding to characteristics of the treatment instrument 2 such as characteristics of the detecting circuit 38 and characteristics of the heater 29.

Moreover, in the embodiments described hereinbefore and so forth, the first electrical energy, or high-frequency power, output from the first output source 31 is supplied to the electrodes 27 and 28 of the end effector 7 and a high-frequency current is caused to flow between the electrodes 27 and 28 through a treatment target, and thereby bipolar treatment is carried out. However, the configuration is not limited thereto. In a certain modification example, a counter electrode that is a separate component from the treatment instrument 2 is disposed in the treatment system 1 and the first electrical energy, or high-frequency power, output from the first output source 31 is supplied to the electrode of the end effector 7 and the counter electrode. Thereby, a high-frequency current flows between the electrode and the counter electrode through a treatment target and monopolar treatment is carried out. Also in this case, the physical quantity Yb relating to the second electrical energy is calculated similarly to the embodiments described hereinbefore and so forth.

Furthermore, in the embodiments described hereinbefore and so forth, an example in which the heater 29 is used as an electrical component to which the second electrical energy is supplied is described. However, the configuration is not limited thereto. In a certain modification example, an ultrasonic transducer is disposed in the treatment instrument 2 as an electrical component instead of the heater 29. In this case, the second electrical energy is supplied from the second output source 35 to the ultrasonic transducer and thereby ultrasonic vibration is generated at the ultrasonic transducer. Then, the generated ultrasonic vibration is transmitted to the end effector 7 and the end effector 7 gives the transmitted ultrasonic vibration to a treatment target as treatment energy. In the present modification example, alternating-current power with any frequency in a predetermined frequency range is supplied to the ultrasonic transducer as the second electrical energy and the frequency of the second electrical energy is low compared with the frequency of the first electrical energy, or high-frequency power, output from the first output source 31.

Moreover, in a certain modification example, a light emitting element is disposed as an electrical component. In the present modification example, alternating-current power with a lower frequency than the first electrical energy, or high-frequency power, is supplied to the light emitting element as the second electrical energy, for example. The light emitting element emits light through supply of the second electrical energy to the light emitting element. Then, a treatment target is subjected to treatment by using laser light or the like generated at the light emitting element.

Furthermore, in another certain modification example, an electric motor is disposed in the treatment instrument 2 as an electrical component to which the second electrical energy is supplied. In the present modification example, direct-current power or alternating-current power with a lower frequency than the first electrical energy, or high-frequency power, is supplied from the second output source 35 to the electric motor as the second electrical energy. Through the supply of the second electrical energy to the electric motor and driving of the electric motor, in certain one example, a flexion joint between the end effector 7 and the shaft 3 is actuated and the end effector 7 makes flexural action with respect to the shaft 3. In addition, in certain another example, through driving of the electric motor, the end effector 7 is actuated as a stapler and a treatment target is punctured by a staple.

Moreover, in the embodiments described hereinbefore and so forth, the electrodes 27, 28, and so forth to which the first electrical energy is supplied and the electrical element such as the heater 29 to which the second electrical energy is supplied are disposed in the same treatment instrument 2. However, the configuration is not limited thereto. In a certain modification example, electrical elements such as the heater 29, an ultrasonic transducer, and electric motor may be disposed in a medical instrument that is a separate portion from the electrodes 27, 28, and so forth to which the first electrical energy is supplied. Also in this case, the physical quantity Yb relating to the second electrical energy is calculated similarly to the embodiments described hereinbefore and so forth.

In the embodiments described hereinbefore and so forth, the first electrical energy is supplied to the electrodes 27, 28 and the second electrical energy is supplied to the electrical element 29 other than the electrodes 27, 28. The processor 25 acquires the first electrical value εa, ηa, or Ya relating to the first electrical energy and the second electrical value εb relating to the second electrical energy, and determines the influence of electrical noise given to the second electrical value εb by the first electrical energy based on the first electrical value εa, ηa, or Ya. The processor 25 calculates the physical quantity Yb relating to the second electrical energy based on the determination result of the influence of the noise and the second electrical value εb.

The disclosed technology of the present application is not limited to the embodiments described hereinbefore and can be variously modified in such a range as not to depart from the gist thereof at the stage of implementation. Furthermore, the respective embodiments may be implemented in such a manner as to be appropriately combined as far as possible and combined effects are obtained in this case. Moreover, the disclosed technologies at various stages are included in the embodiments described hereinbefore and various disclosed technologies can be extracted through an appropriate combination in multiple constituent elements disclosed.

In sum, one aspect of the disclosed technology is directed to a control apparatus of a treatment system having respective first and second output sources that supplies respective high-frequency power defined by a first electrical energy and a second electrical energy. The control apparatus comprises a processor configured to acquire a first value based on the first electrical energy and a second value based on the second electrical energy. Based on the first value, to determine influence given to the second value by the first electrical energy. Based on the influence given to the second value by the first electrical energy and the second value based on the second electrical energy, to adjust an output instruction value to the second output source.

The processor substitutes the second value into a transform function or a transform table to carry out arithmetic operation and substitutes the first value into a noise correction function or a noise correction table to carry out arithmetic operation. The processor adjusts the output instruction value to the second output source by subtracting an arithmetic operation result using the noise correction function or the noise correction table from an arithmetic operation result using the transform function or the transform table. The processor selects one transform function corresponding to the first value from a plurality of transform functions or selects one transform table corresponding to the first value from a plurality of transform tables based on the first value. The processor adjusts the output instruction value to the second output source by substituting the second value into the transform function or the transform table selected to carry out arithmetic operation. The processor acquires any of a detected value being detected regarding the first electrical energy as the first value, an instruction value in an output instruction of the first electrical energy, and a physical quantity relating to the first electrical energy. The processor acquires a detected value detected regarding the second electrical energy as the second value. The processor causes the second output source to output alternating-current power as the second electrical energy. The processor causes the second output source to output the alternating-current power at a lower frequency than the high-frequency power that is the first electrical energy. The processor causes the high-frequency power to be supplied at a frequency that is equal to or higher than 300 kHz and is equal to or lower than 1 MHz and causes the alternating-current power to be supplied at a frequency lower than 200 kHz. The second electrical energy causes a heater to generate heat or causes an ultrasonic transducer to generate ultrasonic vibration.

Another aspect of the disclosed technology is directed to a treatment system comprises a control apparatus having respective first and second output sources that supplies respective high-frequency power defined by a first electrical energy and a second electrical energy. The control apparatus includes a processor configured to acquire a first value based on the first electrical energy and a second value based on the second electrical energy. Based on the first value, the processor determines influence given to the second value by the first electrical energy. Based on the influence given to the second value by the first electrical energy and the second value based on the second electrical energy, to adjust an output instruction value to the second output source. An electrode is configured to receive the first electrical energy and an electrical component is configured to receive the second electrical energy.

The electrical component includes a heater that generates heat by being supplied with the second electrical energy or an ultrasonic transducer that generates ultrasonic vibration by being supplied with the second electrical energy. The treatment system further comprises an end effector configured to contain the electrode and the electrical component. A tubular shaft extends from the end effector. A first electrical path extends inside the tubular shaft and forms a supply path of the first electrical energy to the electrode. A second electrical path extends inside the tubular shaft and forms a supply path of the second electrical energy to the electrical component. The control apparatus is configured to properly calculate a physical quantity relating to the respective first and second electrical energies even when an electrical noise is generated in a state in which multiple electrical energies are simultaneously supplied.

A further aspect of the disclosed technology is directed to a method of operating a treatment system having respective first and second output sources that supplies respective high-frequency power defined by a first electrical energy and a second electrical energy. The method comprises acquiring a first value based on the first electrical energy and a second value based on the second electrical energy: determining influence given to the second value by the first electrical energy based on the first value and adjusting an output instruction value to the second output source based on the influence given to the second value by the first electrical energy and the second value based on the second electrical energy. The second electrical energy causes a heater to generate heat or causes an ultrasonic transducer to generate ultrasonic vibration.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. A control apparatus of a treatment system having respective first and second output sources that supplies respective high-frequency power defined by a first electrical energy and a second electrical energy, the control apparatus comprising:
a processor configured
to acquire a first value based on the first electrical energy and a second value based on the second electrical energy,
based on the first value, to determine influence given to the second value by the first electrical energy, and
based on the influence given to the second value by the first electrical energy and the second value based on the second electrical energy, to adjust an output instruction value to the second output source.

2. The control apparatus of claim 1, wherein
the processor substitutes the second value into a transform function or a transform table to carry out arithmetic operation and substitutes the first value into a noise correction function or a noise correction table to carry out arithmetic operation, and
the processor adjusts the output instruction value to the second output source by subtracting an arithmetic operation result using the noise correction function or the noise correction table from an arithmetic operation result using the transform function or the transform table.

3. The control apparatus of claim 1, wherein
the processor selects one transform function corresponding to the first value from a plurality of transform functions or selects one transform table corresponding to the first value from a plurality of transform tables based on the first value, and
the processor adjusts the output instruction value to the second output source by substituting the second value into the transform function or the transform table selected to carry out arithmetic operation.

4. The control apparatus of claim 1, wherein
the processor acquires any of a detected value being detected regarding the first electrical energy as the first value, an instruction value in an output instruction of the first electrical energy, and a physical quantity relating to the first electrical energy.

5. The control apparatus of claim 1, wherein
the processor acquires a detected value detected regarding the second electrical energy as the second value.

6. The control apparatus of claim 1, wherein
the processor causes the second output source to output alternating-current power as the second electrical energy.

7. The control apparatus of claim 6, wherein
the processor causes the second output source to output the alternating-current power at a lower frequency than the high-frequency power that is the first electrical energy.

8. The control apparatus of claim 7, wherein
the processor causes the high-frequency power to be supplied at a frequency that is equal to or higher than 300 kHz and is equal to or lower than 1 MHz and causes the alternating-current power to be supplied at a frequency lower than 200 kHz.

9. The control apparatus of claim 1, wherein
the second electrical energy causes a heater to generate heat or causes an ultrasonic transducer to generate ultrasonic vibration.

10. A treatment system comprising:
A control apparatus having respective first and second output sources that supplies respective high-frequency power defined by a first electrical energy and a second electrical energy, the control apparatus includes
a processor configured
to acquire a first value based on the first electrical energy and a second value based on the second electrical energy,
based on the first value, to determine influence given to the second value by the first electrical energy, and
based on the influence given to the second value by the first electrical energy and the second value based on the second electrical energy, to adjust an output instruction value to the second output source;
an electrode configured to receive the first electrical energy; and
an electrical component configured to receive the second electrical energy.

11. The treatment system of claim 10, wherein
the electrical component includes a heater that generates heat by being supplied with the second electrical energy or an ultrasonic transducer that generates ultrasonic vibration by being supplied with the second electrical energy.

12. The treatment system of claim 10 further comprising:
an end effector configured to contain the electrode and the electrical component;
a tubular shaft extends from the end effector;
a first electrical path extends inside the tubular shaft and forms a supply path of the first electrical energy to the electrode; and
a second electrical path extends inside the tubular shaft and forms a supply path of the second electrical energy to the electrical component.

13. The treatment system of claim 10, wherein the control apparatus configured to properly calculates a physical quantity relating to the respective first and second electrical energies even when an electrical noise is generated in a state in which multiple electrical energies are simultaneously supplied.

14. A method of operating a treatment system having respective first and second output sources that supplies respective high-frequency power defined by a first electrical energy and a second electrical energy, the method comprising:
acquiring a first value based on the first electrical energy and a second value based on the second electrical energy;
determining influence given to the second value by the first electrical energy based on the first value; and
adjusting an output instruction value to the second output source based on the influence given to the second value by the first electrical energy and the second value based on the second electrical energy.

15. The control method of a treatment system of claim 14, wherein
the second electrical energy causes a heater to generate heat or causes an ultrasonic transducer to generate ultrasonic vibration.
